(19) United States
(12) Patent Application Publication
(10) Pub. No.: US 2026/0137710 A1
(43) Pub. Date: May 21, 2026

(54) PHARMACEUTICAL USE OF NUCLEOSIDE COMPOUND

(71) Applicant: HENAN GENUINE BIOTECH CO., LTD., Henan (CN)

(72) Inventors: Zhaoyang WANG, Henan (CN); Pan LI, Henan (CN); Limin JIA, Henan (CN)

(21) Appl. No.: 19/126,695

(22) PCT Filed: Oct. 27, 2023
(86) PCT No.: PCT/CN2023/127028

§ 371 Date: May 2, 2025

(30) Foreign Application Priority Data
Nov. 2, 2022 (CN) 202211367238.9

(51) Int. Cl. A61K 31/7076; A61K 31/7072; A61P 31/20

(52) U.S. Cl. CPC A61K 31/7076; A61K 31/7072; A61P 31/20

(57) ABSTRACT

Provided is pharmaceutical use of a nucleoside compound, specifically use of compound represented by formula (I) or a pharmaceutically acceptable salt thereof in the preparation of a drug for preventing or treating an infectious disease of orthopoxvirus. The compound represented by formula (I) has a significant inhibitory effect on a vaccinia virus, a monkeypox virus, a cowpox virus, and a smallpox virus.

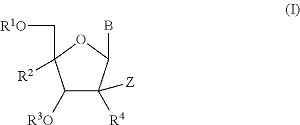

### RELATED APPLICATIONS

The present application is a U.S. National Phase of International Application Number PCT/CN2023/127028 filed on Oct. 27, 2023, which claims priority to Chinese Application Number 202211367238.9 filed on Nov. 2, 2022.

### TECHNICAL FIELD

The present disclosure relates to use of a nucleoside compound in the preparation of a drug for preventing or treating an orthopoxvirus infectious disease.

### BACKGROUND

Most viruses of the genus Orthopoxvirus in the Poxviridae family cause systemic exanthema.

Smallpox virus is the pathogen of smallpox in humans. The initial symptoms after infection with smallpox virus include a high fever, fatigue, a headache, a rapid heartbeat, and back pain, followed by characteristic smallpox rash prominently distributed on the face, arms, and legs. In the early stage of rash, there would also be reddish lumpy areas that accompany the rash. The lesions begin to suppurate and scab over after a few days, then slowly develop into scabies, and subsequently fall off slowly.

Symptoms in humans infected with monkeypox virus are similar to those observed in patients infected with viruses such as smallpox virus in the past. This virus can be transmitted from animals to humans through direct close contact or spread from person to person. The main routes of infection include blood and body fluids. On May 20, 2022, there were more than 100 confirmed and suspected cases of monkeypox in Europe. On May 29, 2022, the World Health Organization (WHO) issued the disease information circular and assessed the global public health risk of monkeypox as “moderate”. On Jun. 24, 2022, the number of confirmed cases of monkeypox worldwide was over 3,200. On Jul. 23, 2022, the WHO classified the monkeypox outbreak as “Public Health Emergency of International Concern”. As of September 2022, the data released by the Centers for Disease Control and Prevention of the United States showed that there were more than 25,000 confirmed cases in the United States. The monkeypox virus is a serious hazard with rapid onset, strong infectivity and a high lethality rate.

Cowpox virus is a virus that may cause mild lesions in cattle. Humans, if infected with this virus, will experience mild discomfort and build resistance to the cowpox virus. Weak in pathogenicity as it is, vaccinia virus is sufficiently potent to induce the body to produce antibodies against diseases. It is bound up with the smallpox virus in serology and immunology and is used as an antigen in the smallpox vaccine.

### SUMMARY

Compound 1 (Azvudine) has already been approved for marketing as a drug for treatment of AIDS and COVID-19, and its safety is guaranteed.

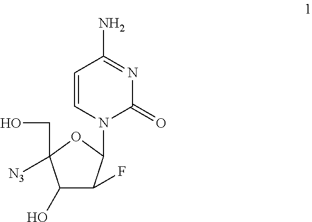 [SMILES: [N-]=[N+]=NC1(CO)OC(n2ccc(N)nc2=O)C(F)C1O]

The present inventors have tested the activity of Compound 1 to inhibit monkeypox virus, smallpox virus, cowpox virus, and vaccinia virus. The results show that Compound 1 has a significant inhibitory effect on all of these viruses.

In view of this, in one aspect of the present disclosure, there is provided use of a compound represented by formula (I) or a pharmaceutically acceptable salt thereof in the preparation of a drug for preventing or treating an orthopoxvirus infectious disease. In another aspect of the present disclosure, there is provided a method for preventing or treating an orthopoxvirus infectious disease, comprising: administering, to a subject in need thereof, a therapeutically or prophylactically effective amount of a compound of formula (I) or a pharmaceutically acceptable salt thereof. In another aspect of the present disclosure, there is provided a pharmaceutical composition, comprising a compound represented by formula (I) or a pharmaceutically acceptable salt thereof and an optional excipient, wherein the pharmaceutical composition is used for preventing or treating an orthopoxvirus infectious disease.

The structure of formula (I) is as follows:

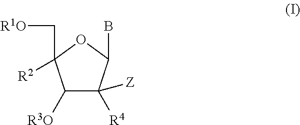

in formula (I),
R¹ is any group that enables the OR¹ group to metabolize in vivo to release a hydroxyl group or form an O-phosphate group; OR¹ is preferably an ester group, R¹ is preferably H, R⁵—CO— or

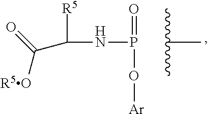

wherein Ar is phenyl, substituted phenyl, naphthyl or substituted naphthyl, and the substituent is selected from C₁₋₆ alkyl, F, Cl, Br, I, CN, N₃, OH, NH₂, OR⁵, and NHR⁵;
R² is selected from: H, azido, C₁-C₆ alkyl (such as methyl or ethyl), C₁-C₆ alkoxy (such as methoxy or ethoxy), C₂-C₆ alkynyl (such as ethynyl), C₂-C₆ alkenyl (such as vinyl), and halogenated C₁-C₆ alkyl (such as 2-chloroethyl, 2-fluoroethyl or trifluoroethyl);
R³ is selected from H, optionally substituted R—CO—, optionally substituted R—O(C═O)—, and optionally substituted RNH—CO—, wherein R is selected from C₁-C₆ alkyl (such as methyl, ethyl, propyl, isopropyl, butyl, isobutyl, pentyl or isopentyl); wherein the substituent is selected from C₁-C₆ alkyl, halogen (such as F or Cl), CN, N₃, and OR⁵;
R⁴ is selected from H, OH, halogen (such as F), C₁-C₆ alkyl (such as methyl or ethyl), and C₁-C₆ alkoxy (such as methoxy or ethoxy);
B is selected from:

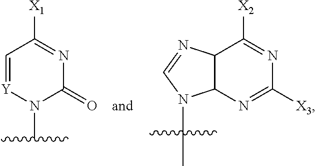

wherein X₁ is selected from —OH, —NH₂, R⁵CONH—, R⁵COO—, and R⁵O(C═O) NH—;
X₂ is selected from —OH, —SH, —NH₂, R⁵COO—, R⁵COS—, R⁵CONH₂—, and R⁵O (C═O) NH—;
X₃ is selected from H, F, —OH, and —NH₂;
Y is selected from CH and N;
Z is H, —OH or F;
R⁵ is selected from H, C₁-C₆ alkyl (such as methyl, ethyl, propyl or isopropyl), C₂-C₆ alkynyl (such as ethynyl), C₂-C₆ alkenyl (such as vinyl), halogenated C₁-C₆ alkyl (such as 2-chloroethyl, 2-fluoroethyl or trifluoroethyl), phenyl optionally substituted with C₁₋₆ alkyl, C₁₋₆ alkoxy, CN, N₃, OH, NH₂ or halogen (such as F, Cl, Br or I), and naphthyl optionally substituted with C₁₋₆ alkyl, C₁₋₆ alkoxy, CN, N₃, OH, NH₂ or halogen (such as F, Cl, Br or I).

Preferably, in formula (I),
R¹ is H or R⁵—CO—;
R² is azido;
R³ is H or R—CO—, wherein R is C₁-C₆ alkyl;
R⁴ is halogen;
B is:

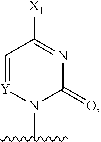

wherein X₁ is-NH₂;
Y is CH;
Z is H;
R⁵ is selected from H and C₁-C₆ alkyl.

The following compounds or pharmaceutically acceptable salts thereof are preferred:

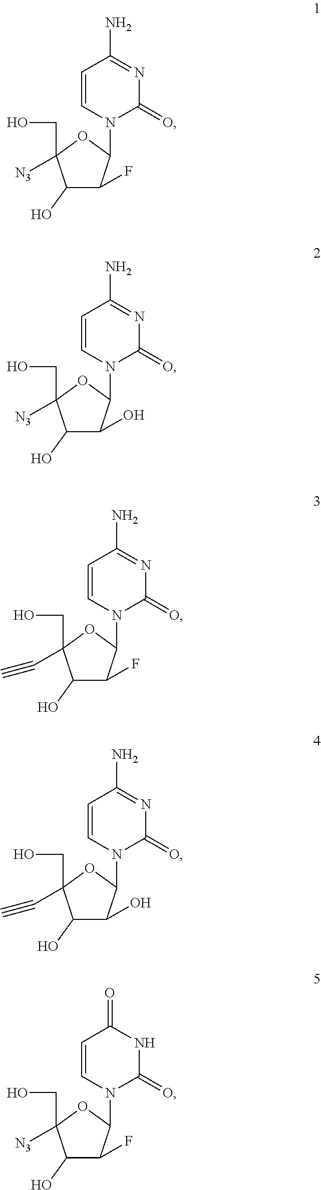 [SMILES: [N-]=[N+]=NC1(CO)OC(n2ccc(N)nc2=O)C(F)C1O ; [N-]=[N+]=NC1(CO)OC(n2ccc(N)nc2=O)C(O)C1O ; C#CC1(CO)OC(n2ccc(N)nc2=O)C(F)C1O ; C#CC1(CO)OC(n2ccc(N)nc2=O)C(O)C1O ; [N-]=[N+]=NC1(CO)OC(n2ccc(=O)[nH]c2=O)C(F)C1O]

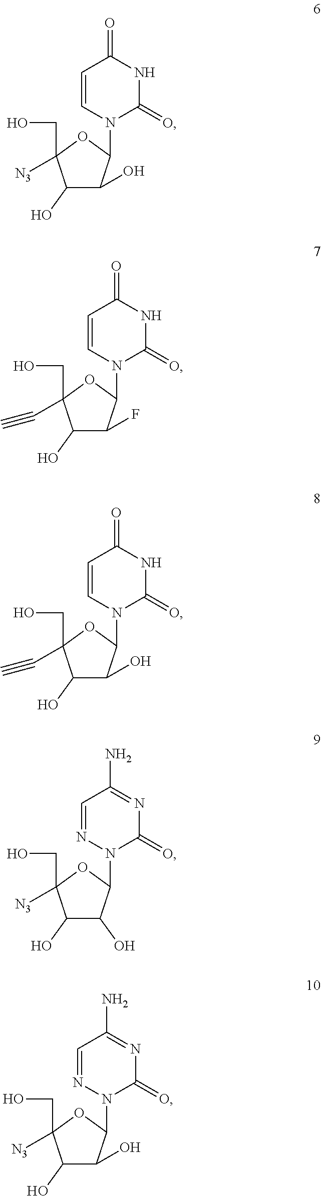 [SMILES: [N-]=[N+]=NC1(CO)OC(n2ccc(=O)[nH]c2=O)C(O)C1O ; C#CC1(CO)OC(n2ccc(=O)[nH]c2=O)C(F)C1O ; C#CC1(CO)OC(n2ccc(=O)[nH]c2=O)C(O)C1O ; [N-]=[N+]=NC1(CO)OC(n2ncc(N)nc2=O)C(O)C1O ; [N-]=[N+]=NC1(CO)OC(n2ncc(N)nc2=O)C(O)C1O]

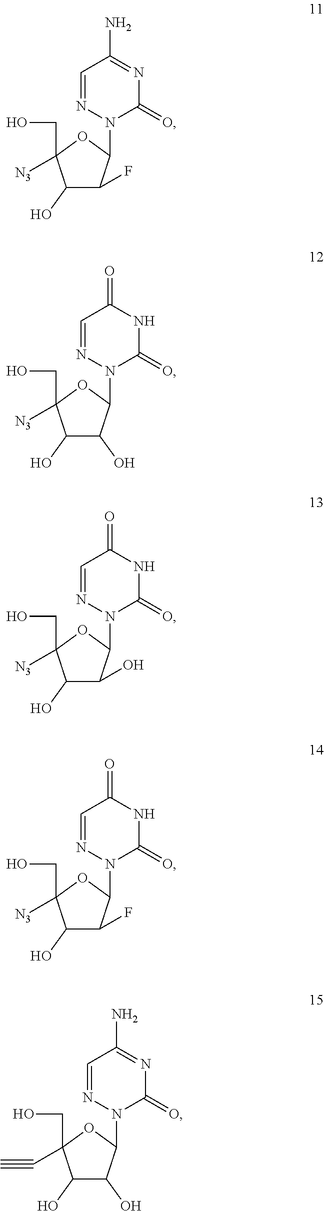 [SMILES: [N-]=[N+]=NC1(CO)OC(n2ncc(N)nc2=O)C(F)C1O ; [N-]=[N+]=NC1(CO)OC(n2ncc(=O)[nH]c2=O)C(O)C1O ; [N-]=[N+]=NC1(CO)OC(n2ncc(=O)[nH]c2=O)C(O)C1O ; [N-]=[N+]=NC1(CO)OC(n2ncc(=O)[nH]c2=O)C(F)C1O ; C#CC1(CO)OC(n2ncc(N)nc2=O)C(O)C1O]

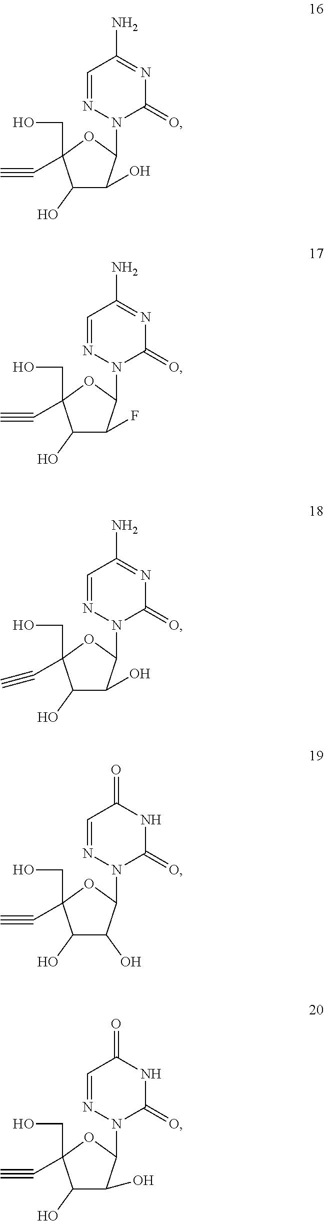 [SMILES: C#CC1(CO)OC(n2ncc(N)nc2=O)C(O)C1O ; C#CC1(CO)OC(n2ncc(N)nc2=O)C(F)C1O ; C#CC1(CO)OC(n2ncc(N)nc2=O)C(O)C1O ; C#CC1(CO)OC(n2ncc(=O)[nH]c2=O)C(O)C1O ; C#CC1(CO)OC(n2ncc(=O)[nH]c2=O)C(O)C1O]

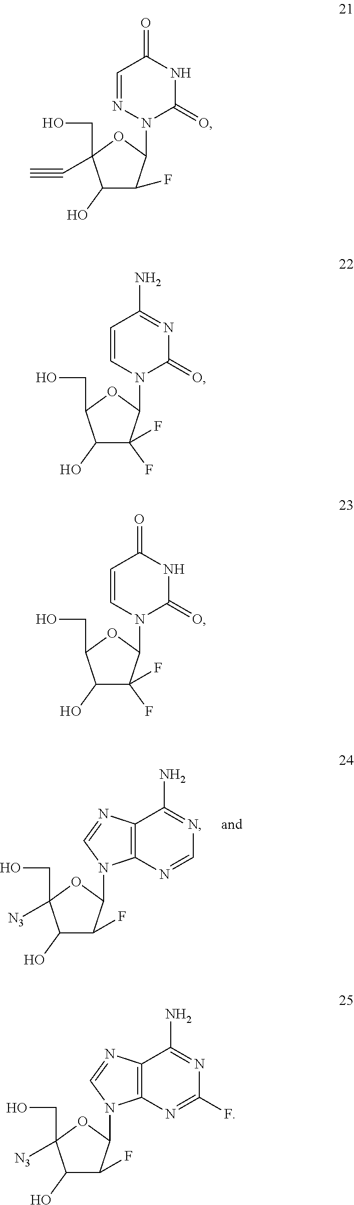 [SMILES: C#CC1(CO)OC(n2ncc(=O)[nH]c2=O)C(F)C1O ; Nc1ccn(C2OC(CO)C(O)C2(F)F)c(=O)n1 ; O=c1ccn(C2OC(CO)C(O)C2(F)F)c(=O)[nH]1 ; [N-]=[N+]=NC1(CO)OC(n2cnc3c(N)ncnc32)C(F)C1O ; [N-]=[N+]=NC1(CO)OC(n2cnc3c(N)nc(F)nc32)C(F)C1O]

The pharmaceutically acceptable salt of the compound of formula (I) includes, but is not limited to, for example, salts formed of the compound of formula (I) with the following acids: hydrochloric acid, hydrobromic acid, sulfamic acid, sulfuric acid, phosphoric acid, nitric acid, formic acid, acetic acid, propionic acid, oxalic acid, glycolic acid, malonic acid, benzoic acid, lactic acid, gluconic acid, citric acid, tartaric acid, succinic acid, fumaric acid, maleic acid, mandelic acid, malic acid, methanesulfonic acid, ethanesulfonic acid, isethionic acid, benzenesulfonic acid, p-toluenesulfonic acid, naphthalenesulfonic acid, naphthalenedisulfonic acid, camphorsulfonic acid, ascorbyl palmitic acid, salicylic acid, sulfosalicylic acid, 2-hydroxy-3-naphthoic acid, phthalic acid, lysine, arginine, glutamic acid, glycine, serine, threonine, alanine, isoleucine, leucine, etc.

The compound of formula (I) may be commercially available or prepared by known methods.

The orthopoxvirus infectious disease used herein is a disease caused by infection with viruses of the genus Orthopoxvirus, including diseases caused by infection of humans or other animals, particularly diseases caused by infection of humans. The orthopoxvirus includes, for example, monkeypox virus, cowpox virus, and smallpox virus.

The effective amount of the compound of formula (I) for preventing or treating the orthopoxvirus infectious disease may be determined by a person skilled in the art based on the information provided herein. For example, the dosage for an adult may be 1 to 500 mg/day, optionally 1 to 50 mg/day, optionally 1 to 20 mg/day, optionally 1 to 10 mg/day, or optionally 5 mg/day. It can be administered in a single dose or in multiple doses.

The mode of administration may be oral or parenteral administration. It may be an immediate-release dosage form, or may be a sustained-release dosage form or a controlled-release dosage form. The specific dosage forms may be various conventional dosage forms in the art. For example, examples of the oral preparation may include tablets, hard or soft capsules, aqueous or oily suspensions, granules, emulsions, syrup or elixirs. Examples of the injection may include injections or powder injections.

### DETAILED DESCRIPTION

The specific embodiments of the present disclosure will be described in detail below. It should be appreciated that the specific embodiments described herein are merely intended to illustrate the present disclosure and not to limit the present disclosure.

### Example: Antiviral Activity Assay on Compound

### 1.1 Cell Lines and Virus Strains

Human foreskin fibroblasts HFF-1 and the virus strains of Vaccinia VP13, Monkeypox virus, Cowpox virus, and Smallpox virus were obtained from American Type Culture Collection (ATCC) and China Center for Type Culture Collection (CCTCC), respectively. The experimental medium was DMEM medium supplemented with 2% fetal bovine serum, 2 mM L-glutamine, 1% non-essential amino acids, 1% dual resistance, and 1% sodium pyruvate.

### 1.2 Compound

The test compound was a solid powder of Compound 1 provided by Henan Genuine Biotech Co., Ltd., and was formulated into a 20 mM stock solution with 100% DMSO. Control compound ST-246 (MedChemExpress, Cat. No.: HY-14805) was provided by WuXi AppTec. The compound was tested at 8 concentrations each for 3-fold serial dilutions and triplicate wells. The starting test concentration of the test compound was 10 μM. The maximum test concentration was listed in Table 1.

TABLE 1
Information of Compounds

| | Molecular | Maximum Test | |
|---|---|---|---|
| Compound | Weight | Concentration | Remarks |
| Compound 1 | 286.22 | 10 μM | Test compound |
| ST-246 | 376.33 | 1 μM and 100 μM* | Control compound |

*Note:
The maximum test concentrations of ST-246 for antiviral activity and cytotoxicity were 1 μM and 100 μM, respectively.

### 1.3 Main Reagent and Instrument

(1) Main instrument: Microplate Reader (Molecule Devices, model: SpectraMax340PC384).
(2) Main reagent: Cell Counting Kit CCK-8 (Shanghai Life iLab Biotech Co., LTD, Cat. No.: AC11L057).

### 2. Experimental Method

ST-246 was a control compound for activity assay. The compound was tested at 8 concentrations each for triplicate wells.

TABLE 2
Experimental Method for Antiviral Activity

| | | Compound Treatment | Positive |
|---|---|---|---|
| Virus | Cells | Period (day)/Experiment | Control |
| Vaccinia/VP13 | HFF-1 | 4/CPE | ST-246 |
| Monkeypox virus | HFF-1 | 4/CPE | ST-246 |
| Cowpox virus | HFF-1 | 4/CPE | ST-246 |
| Smallpox virus | HFF-1 | 4/CPE | ST-246 |

Day 1: HFF-1 cells were inoculated into a 96-well test plate at a density of 6,000 cells in 100 μL per well, and cultured overnight in an incubator at 5% CO₂, 37° C.

Day 2: 50 μL of serially diluted compound (8 concentrations, 3-fold dilution, triplicate wells) and 50 μL of viruses (at an inoculation volume of MOI=0.1) were added to each well, respectively. Cell control (cells, no compound treatment and virus infection), virus control (cells infected with virus, no compound treatment) and medium control (medium only) were set up. The total volume of cell medium was 200 μL per well. The final concentrations of DMSO in the media were 0.5%, respectively. The cells were cultured in an incubator at 5% CO₂, 37° C. for 4 days.

Day 6: Cell viability was detected by Cell Counting Kit CCK8. The antiviral activity and cytotoxicity of the test samples were expressed by the inhibition rate (%) of the cytopathic effect caused by the virus at different concentrations and the cell viability (%), respectively. They were calculated by the following formulae:

Inhibition Rate (%)=(test well reading−virus control average)/(cell control average−virus control average)×100

Cell Viability (%)=(test well reading−medium control average)/(cell control average−medium control average)×100

EC₅₀ and CC₅₀ values were calculated by the Prism software (version 7), and the curve of inhibition was fitted by the following method: log (inhibitor) vs. response—Variable slope of GraphPad Prism.

### 3. Results and Discussion

In the cytopathic effect assay, the positive control compound ST-246 showed the expected antiviral activity and cytotoxicity, indicating the reliability of the experiment. The antiviral activity and cytotoxicity results of the test compound and the control compound were summarized in Table 3.

TABLE 3
Antiviral Activity Results of Compound

| No. | Virus | CPD ID | EC₅₀ (μM) | CC₅₀ (μM) |
|---|---|---|---|---|
| 1 | Vaccinia VP13 | Compound 1 | 0.57 | >10 |
| | | ST-246 | 0.04 | >100 |
| 2 | Monkeypox virus | Compound 1 | 0.32 | >10 |
| | | ST-246 | 0.03 | >100 |
| 3 | Cowpox virus | Compound 1 | 0.75 | >10 |
| | | ST-246 | 0.05 | >100 |
| 4 | Smallpox virus | Compound 1 | 1.2 | >10 |
| | | ST-246 | 0.07 | >100 |

The results showed that Compound 1 had significant inhibitory effects on Vaccinia virus, Monkeypox virus, Cowpox virus, and Smallpox virus, and the EC₅₀ values were 0.57, 0.32, 0.75, and 1.20 μM, respectively. While the antiviral activity of Compound 1 was weaker than that of ST-246, Compound 1 has been approved for marketing as a drug for treatment of AIDS and COVID-19, and its safety is guaranteed. Therefore, Compound 1 has greater potential for clinical application.

1-6. (canceled)

7. A method for preventing or treating an orthopoxvirus infectious disease, including giving an effective amount of a compound represented by formula (I) or a pharmaceutically acceptable salt thereof to the subject in need, 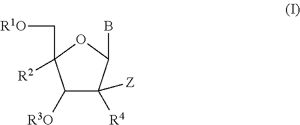
wherein in formula (I),
R¹ is H, R⁵—CO— or 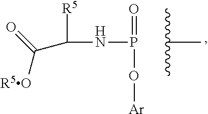
wherein Ar is phenyl, substituted phenyl, naphthyl or substituted naphthyl, wherein a substituent is selected from C₁₋₆ alkyl, F, Cl, Br, I, CN, N₃, OH, NH₂, OR⁵, and NHR⁵;
R² is H, azido, C₁-C₆ alkyl (such as methyl or ethyl), C₁-C₆ alkoxy (such as methoxy or ethoxy), C₂-C₆ alkynyl (such as ethynyl), C₂-C₆ alkenyl (such as vinyl), or halogenated C₁-C₆ alkyl (such as 2-chloroethyl, 2-fluoroethyl or trifluoroethyl);
R³ is H, optionally substituted R—CO—, optionally substituted R—O(C═O)— or optionally substituted RNH—CO—, wherein R is C₁-C₆ alkyl (such as methyl, ethyl, propyl, isopropyl, butyl, isobutyl, pentyl or isopentyl), wherein a substituent is selected from C₁-C₆ alkyl, halogen (such as F or Cl), CN, N₃, and OR⁵;
R⁴ is H, OH, halogen (such as F), C₁-C₆ alkyl (such as methyl or ethyl) or C₁-C₆ alkoxy (such as methoxy or ethoxy);
B is selected from: 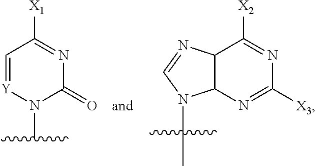
wherein X₁ is —OH, —NH₂, R⁵CONH—, R⁵COO— or R⁵O(C═O)NH—;
X₂ is OH, SH, NH₂, R⁵COO—, R⁵COS—, R⁵CONH₂— or R⁵O(C═O NH—;
X₃ is H, F, OH or NH₂;
Y is CH or N;
Z is H, OH or F;
R⁵ is selected from H, C₁-C₆ alkyl (such as methyl, ethyl, propyl or isopropyl), C₂-C₆ alkynyl (such as ethynyl), C₂-C₆ alkenyl (such as vinyl), halogenated C₁-C₆ alkyl (such as 2-chloroethyl, 2-fluoroethyl or trifluoroethyl), phenyl optionally substituted with C₁₋₆ alkyl, C₁₋₆ alkoxy, CN, N₃, OH, NH₂ or halogen (such as F, Cl, Br or I), and naphthyl optionally substituted with C₁₋₆ alkyl, C₁₋₆ alkoxy, CN, N₃, OH, NH₂ or halogen (such as F, Cl, Br or I).

8. The method according to claim 7, wherein in formula (I),
R¹ is H or R⁵—CO—;
R² is azido;
R³ is H or R—CO—, wherein R is C₁-C₆ alkyl;
R⁴ is halogen;
B is: 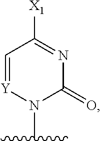
wherein X₁ is —NH₂;
Y is CH;
Z is H;
R⁵ is selected from H and C₁-C₆ alkyl.

9. The method according to claim 7, wherein the compound represented by formula (I) is the following compound:

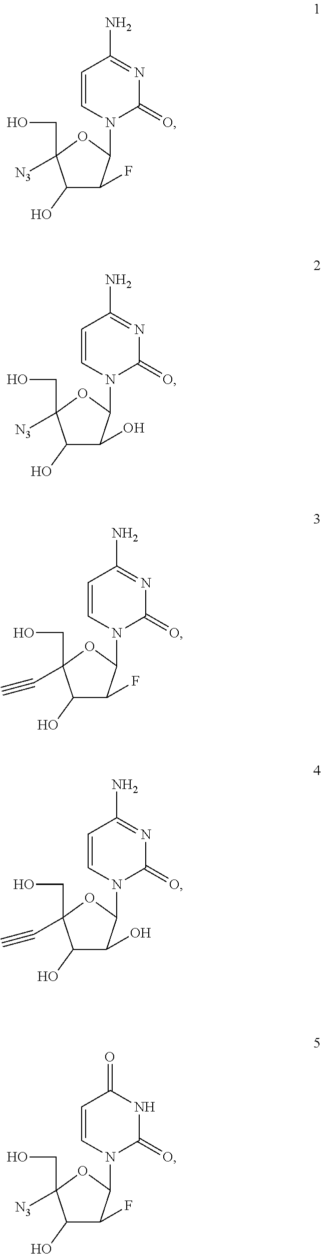 [SMILES: [N-]=[N+]=NC1(CO)OC(n2ccc(N)nc2=O)C(F)C1O ; [N-]=[N+]=NC1(CO)OC(n2ccc(N)nc2=O)C(O)C1O ; C#CC1(CO)OC(n2ccc(N)nc2=O)C(F)C1O ; C#CC1(CO)OC(n2ccc(N)nc2=O)C(O)C1O ; [N-]=[N+]=NC1(CO)OC(n2ccc(=O)[nH]c2=O)C(F)C1O]

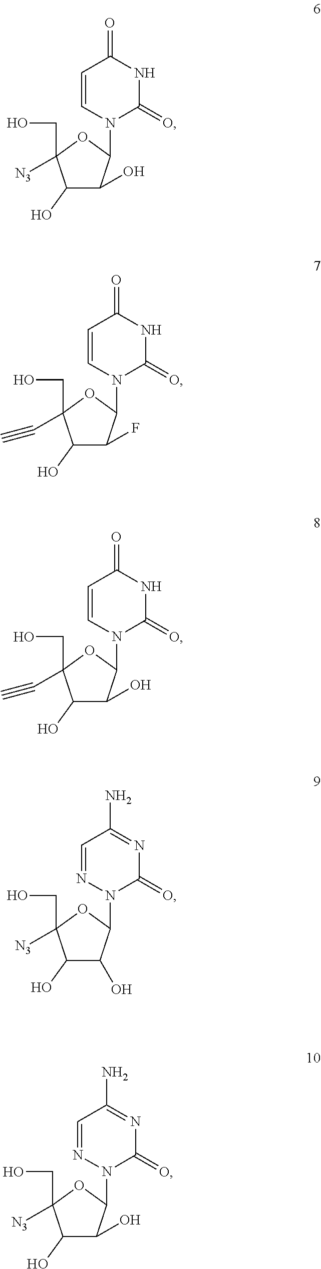 [SMILES: [N-]=[N+]=NC1(CO)OC(n2ccc(=O)[nH]c2=O)C(O)C1O ; C#CC1(CO)OC(n2ccc(=O)[nH]c2=O)C(F)C1O ; C#CC1(CO)OC(n2ccc(=O)[nH]c2=O)C(O)C1O ; [N-]=[N+]=NC1(CO)OC(n2ncc(N)nc2=O)C(O)C1O ; [N-]=[N+]=NC1(CO)OC(n2ncc(N)nc2=O)C(O)C1O]

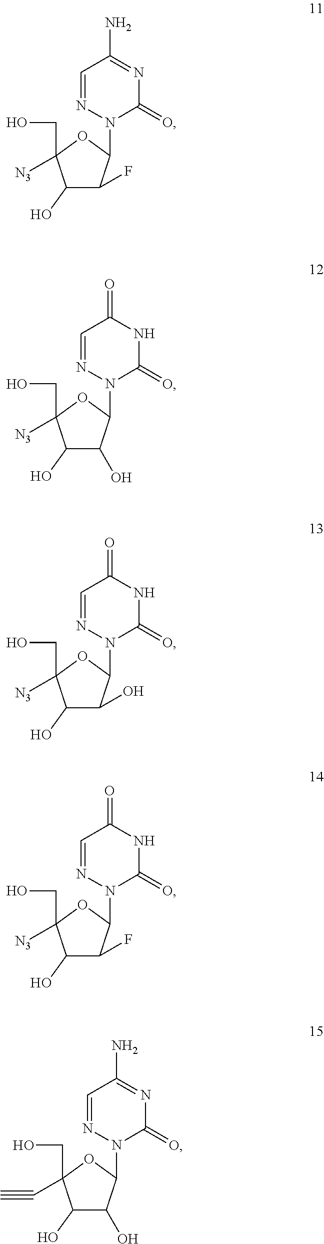 [SMILES: [N-]=[N+]=NC1(CO)OC(n2ncc(N)nc2=O)C(F)C1O ; [N-]=[N+]=NC1(CO)OC(n2ncc(=O)[nH]c2=O)C(O)C1O ; [N-]=[N+]=NC1(CO)OC(n2ncc(=O)[nH]c2=O)C(O)C1O ; [N-]=[N+]=NC1(CO)OC(n2ncc(=O)[nH]c2=O)C(F)C1O ; C#CC1(CO)OC(n2ncc(N)nc2=O)C(O)C1O]

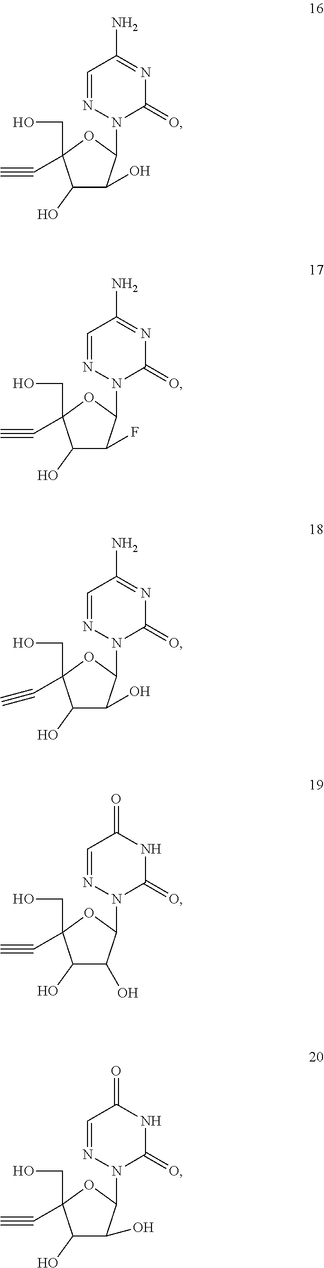 [SMILES: C#CC1(CO)OC(n2ncc(N)nc2=O)C(O)C1O ; C#CC1(CO)OC(n2ncc(N)nc2=O)C(F)C1O ; C#CC1(CO)OC(n2ncc(N)nc2=O)C(O)C1O ; C#CC1(CO)OC(n2ncc(=O)[nH]c2=O)C(O)C1O ; C#CC1(CO)OC(n2ncc(=O)[nH]c2=O)C(O)C1O]

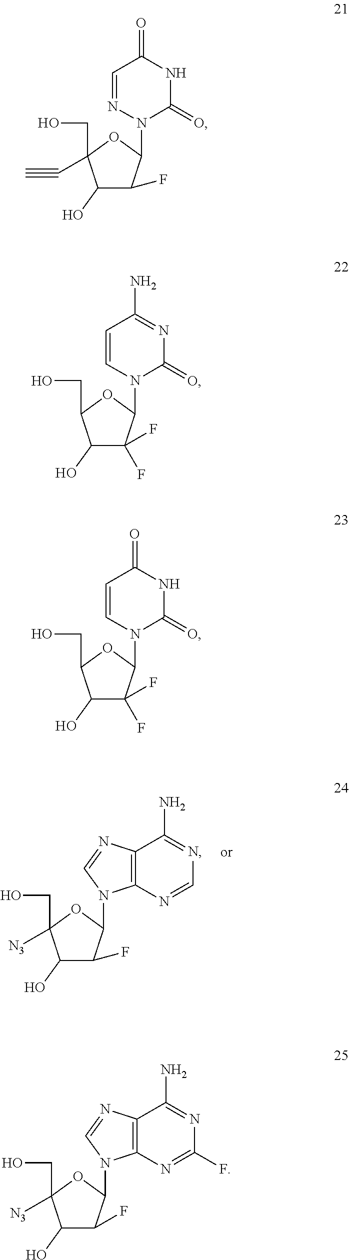 [SMILES: C#CC1(CO)OC(n2ncc(=O)[nH]c2=O)C(F)C1O ; Nc1ccn(C2OC(CO)C(O)C2(F)F)c(=O)n1 ; O=c1ccn(C2OC(CO)C(O)C2(F)F)c(=O)[nH]1 ; [N-]=[N+]=NC1(CO)OC(n2cnc3c(N)ncnc32)C(F)C1O ; [N-]=[N+]=NC1(CO)OC(n2cnc3c(N)nc(F)nc32)C(F)C1O]

10. The method according to claim 7, wherein the pharmaceutically acceptable salt of the compound represented by formula (I) comprises a salt formed of the compound represented by formula (I) with the following acid: hydrochloric acid, hydrobromic acid, sulfamic acid, sulfuric acid, phosphoric acid, nitric acid, formic acid, acetic acid, propionic acid, oxalic acid, glycolic acid, malonic acid, benzoic acid, lactic acid, gluconic acid, citric acid, tartaric acid, succinic acid, fumaric acid, maleic acid, mandelic acid, malic acid, methanesulfonic acid, ethanesulfonic acid, isethionic acid, benzenesulfonic acid, p-toluenesulfonic acid, naphthalenesulfonic acid, naphthalenedisulfonic acid, camphorsulfonic acid, ascorbyl palmitic acid, salicylic acid, sulfosalicylic acid, 2-hydroxy-3-naphthoic acid, phthalic acid, lysine, arginine, glutamic acid, glycine, serine, threonine, alanine, isoleucine or leucine.

11. The method according to claim 7, wherein the orthopoxvirus is monkeypox virus, cowpox virus or smallpox virus, and the orthopoxvirus infectious disease includes a disease caused by infection of humans or other animals.

12. The method according to claim 7, wherein a dosage form of the drug is an immediate-release dosage form, a sustained-release dosage form or a controlled-release dosage form, such as a tablet, a hard or soft capsule, an aqueous or oily suspension, a granule, an emulsion, a syrup, an elixir, an injection or a powder injection.

13. The method according to claim 10, wherein the orthopoxvirus is monkeypox virus, cowpox virus or smallpox virus, and the orthopoxvirus infectious disease includes a disease caused by infection of humans or other animals.

14. The method according to claim 10, wherein a dosage form of the drug is an immediate-release dosage form, a sustained-release dosage form or a controlled-release dosage form, such as a tablet, a hard or soft capsule, an aqueous or oily suspension, a granule, an emulsion, a syrup, an elixir, an injection or a powder injection.

15. The method according to claim 11, wherein a dosage form of the drug is an immediate-release dosage form, a sustained-release dosage form or a controlled-release dosage form, such as a tablet, a hard or soft capsule, an aqueous or oily suspension, a granule, an emulsion, a syrup, an elixir, an injection or a powder injection.
